(19) United States
(12) Patent Application Publication
(10) Pub. No.: US 2008/0317727 A1
(43) Pub. Date: Dec. 25, 2008

(54) Method of skin treatment for rosacea using epidermal growth factor

(76) Inventors: Hannah Naomi Sivak, Gilbert, AZ (US); Artist Cloutier, Portland, OR (US)

Correspondence Address: HANNAH N. SIVAK, 1213 W. SEASCAPE DR, GILBERT, AZ, 85233, US

(21) Appl. No.: 11/416,044

(22) Filed: May 2, 2006

(51) Int. Cl. A61K 38/18; A61K 38/44; A61K 38/05; A61K 31/355; A61K 31/12; A61K 31/015; A61K 31/375

(52) U.S. Cl. 424/94.4; 514/12; 514/18; 514/458; 514/474; 514/690; 514/763

(57) ABSTRACT

Compositions and methods for the prevention and treatment of skin and ocular rosacea using epidermal growth factor and an acceptable carrier are disclosed.

### BACKGROUND OF THE INVENTION

Rosacea is a chronic, progressive facial skin disorder that affects more than ten million Americans, with almost half of the sufferers aged between 30 and 50 years old. The disease has been called “the Celtic curse” because it affects more often people of Northern European descent. Women are more likely to suffer rosacea of the milder form, and men more frequently have the severe form, which involves deformity of the nose. Rosacea nearly always appears on sun damaged skin.

Except for cases precipitated by use of steroids, the causes of rosacea are unknown. Because we do not know what causes the disease, treatment is limited to topical and oral medications that combat associated inflammation, bumps, and pimples. Currently, the mainstay of treatment also consists of making significant lifestyle changes in order to avoid facial capillary vasodilation (flushing and blushing), in order to slow progression.

Rosacea develops in stages and is characterized by “twitchy” blood vessels, i.e., subcutaneous blood vessels that are too sensitive. Many different factors can start flushing and blushing episodes followed by redness of the skin caused by congestion of the capillaries and chronic dilation of capillaries causing elevated dark red blotches on the skin. Rosacea patients may develop severe sebaceous gland growth that is accompanied by papules, pustules, cysts, and nodules. Inflammatory lesions develop in the areas of erythema and may look like acne, but in rosacea there are no comedones, the primary event in acne. Some patients experience burning or stinging sensations.

Ocular symptoms occur in many patients with rosacea, most commonly in combination with skin symptoms, but occasionally alone. Eye symptoms may include foreign body sensation and burning, telangiectasia and conjunctivitis.

Rhinophyma, a distressing symptom that consists in the hyperplasia of the soft tissues of the nose, tends to occur late in the course of rosacea, most commonly in middle-aged men.

Epidermal growth factor is a small protein composed of 53 amino acids with a molecular weight of 6,200. EGF directly stimulates the proliferation of epidermal cells, and this stimulatory action of EGF does not depend on other systemic or hormonal influences. Cells will respond to EGF only if they have receptors on the cell membrane that recognize the factor. The growth factor is produced by other cells that may be near or far from the target cell. The binding of the growth factor to the receptor initiates a cascade of molecular events that will eventually lead, among other effects, to cell division. EGF is used to accelerate healing of skin and cornea.

Under certain conditions, endothelium contracts in response to epidermal growth factor (Florian and Watts, 1999). This effect is prevented by 4,5-dianilinophthalimide (an inhibitor of the EGF receptor tyrosine kinase), by PD-098059 (an inhibitor of the kinase of the EGF-activated protein kinase), and by diltiazem (an inhibitor of the L-type voltage-gated calcium channel), confirming that the endothelium contraction was caused by the growth factor.

Whatever the underlying causes of rosacea, its manifestations are initiated by the dilation of blood vessels. If epidermal growth factor could contract the capillaries involved in skin and eye rosacea, subsequent symptoms could be prevented.

Application of a gel containing distilled water, hyaluronic acid, and human epidermal growth factor (transgenic) in a 0.04% concentration abolished the usual symptoms in a woman suffering from chronic skin rosacea. After 6 weeks of application, the woman reported no reappearance of the symptoms and that her skin is still clear.

### DESCRIPTION OF THE INVENTION

The present invention provides a method and composition for a preventive regimen and/or therapy of dermal and ocular rosacea based upon the topical application to exposed or affected skin areas of at least one active agent, in association with a dermatologically acceptable carrier or vehicle. This invention is based upon the finding that epidermal growth factor can have a vasoconstrictor effect.

These and other objectives are accomplished by the present invention, which provides methods and compositions for the prevention and/or treatment of skin affected by rosacea, by applying topically to the exposed or affected skin areas an effective amount of epidermal growth factor, preferably in a dermatologically acceptable carrier.

The present invention also provides methods and compositions for the prevention and/or treatment of ocular rosacea, by applying to the affected eye an effective amount of epidermal growth factor, preferably in an acceptable carrier.

As used herein, the term “epidermal growth factor” encompasses growth factors purified from natural sources such as human urine or obtained by genetic engineering means, i.e., by over-expression of a protein foreign to the organism used for its production, and to proteins of different molecular weight and/or sequence that are considered growth factors and are capable of exerting a similar action of vascular tissue. Human epidermal growth factor obtained by overexpression in yeast or bacteria is preferred because they are less expensive than those obtained by purification from natural materials such as rice roots.

Many embodiments incorporate at least one other active ingredient with the epidermal growth factor. These include natural or synthetic antioxidant molecules such as reduced glutathione, tocotrienols, vitamin E, ascorbic acid, astaxanthin, and/or lycopene. Other desirable ingredients are proteins capable of alleviating oxidative stress such as catalase, thioredoxin and/or superoxide dismutase.

In the preferred practice of the invention, an epidermal growth factor preparation is applied in admixture with a dermatologically acceptable carrier or vehicle (e.g., as a lotion, cream, ointment, serum) so as to facilitate topical application and, in some cases, provide additional therapeutic effects as might be brought about by moisturizing the affected skin areas. As noted, other ingredients are advantageously included in the compositions.

The amount of epidermal growth factor necessary to bring about prevention and/or therapeutic treatment of rosacea skin is not fixed, and is dependent upon the source, purity and activity of the protein employed, the amount and type of any additional ingredients used, particularly those that appear to exhibit synergistic effects, the skin type of the user, and, where present, the severity and extent of skin damage. Generally, the epidermal growth factor or composition containing it is topically applied in effective amounts to skin areas which are affected by rosacea or have a predisposition to show symptoms of rosacea.

In one embodiment, the composition contains from about 0.0001% to about 0.05% (weight per volume), preferably from more than 0.01% to about 0.04% epidermal growth factor.

While the carrier for epidermal growth factor can be very simple (such as saline solution), it is generally preferred that the carrier be a composition that will facilitate topical application, and particularly one which will form a film or layer on the skin to which it is applied so as to localize the active ingredient. Many such compositions are known in the art, and can take the form of lotions, creams, gels, etc. Typical compositions include lotions containing water and/or alcohols and emollients such as natural oils and waxes, silicone oils, hyaluronic acid, glyceride derivatives, fatty acids or fatty acid esters or alcohols or alcohol ethers, lanolin and derivatives, polyhydric alcohols or esters, wax esters, sterols, phospholipids and the like, and generally also emulsifiers (nonionic, cationic or anionic), although some of the emollients inherently possess emulsifying properties. These same general ingredients can be formulated into a cream rather than a lotion, or into gels, or into solid sticks by utilization of different proportions of the ingredients and/or by inclusion of thickening agents such as gums or other forms of hydrophilic colloids. Such compositions are referred to herein as dermatologically-acceptable carriers.

Many preferred embodiments of this invention contain at least one or two, and sometimes several, other active ingredients in addition to epidermal growth factor, provided that the ingredients are not acids present in concentrations high enough to denature and inactivate the proteins.

Reduced glutathione, tocotrienol, lycopene, astaxanthin, ascorbic acid, and/or vitamin E may also be added to the epidermal growth factor composition, alone or in combination with other ingredients in some embodiments.

A similar approach is used for ocular rosacea: application of saline solution containing human epidermal growth factor (transgenic) in a 0.04% concentration.

In terms of a possible explanation for the effectiveness of the active ingredients in the prevention or treatment of damage to the skin, it is noted that epidermal growth factor may be acting through its effect as vasoconstrictor. Just like in the living cell, where a number of antioxidants work in a concerted fashion, some embodiments of this invention also use the synergistic effect of antioxidants.

Having described the invention with reference to particular compositions, theories of effectiveness, it will be apparent to those of skill in the art that it is not intended that the invention be limited by such illustrative embodiments or mechanisms, and that modifications can be made without departing from the scope or spirit of the invention, as defined by the appended claims. It is intended that all modifications and variations be included within the scope of the invention. The claims are meant to cover the claimed components and steps in any sequence which is effective to meet the objectives there intended, unless the context specifically indicates the contrary.

### RELEVANT REFERENCES

Florian, J. A. and Watts, S. W. Epidermal growth factor: a potent vasoconstrictor in experimental hypertension. Am. J. Physiol. Heart Circ. Physiol. (1999) 276, H976-H83.
Florian, J. A. et al. (2001) Mineralocorticoids upregulate arterial contraction to epidermal growth factor. Am J Physiol Regul Integr Comp Physiol Vol. 281, Issue 3, R878-R886,

We claim:

1. A method for the prevention and treatment of rosacea skin which consists of applying a composition containing epidermal growth factor in a dermatologically acceptable carrier to the affected skin area.

2. A method for the treatment of ocular rosacea which consists of applying a composition containing epidermal growth factor to the affected eye.

3. A method in accordance with claims 1 or 2, wherein said compositions further comprise one or more additional ingredients selected from the group consisting of tocotrienols, vitamin E, ascorbic acid, thioredoxin, superoxide dismutase, catalase, astaxanthin, lycopene, reduced glutathione.
